SEQUENCE LISTING (10 sequences)

SEQ ID NO: 1 - protein, 107 aa, Artificial Sequence
  synthetic peptide antibody light chain variable region
DIQMTQSPASLSASLGETVTIECRASEDIYNALAWYQQKPGKSPQLLIYNTDTLHTGVPSRFSGSGSGTQYSLKINSLQSEDVASYFCQHYFHYPRTFGGGTKLELK

SEQ ID NO: 2 - protein, 122 aa, Artificial Sequence
  synthetic peptide antibody heavy chain variable region
QVQLKESGPGLVQPSQTLSLTCTVSGFSLTNNNVNWVRQATGRGLEWMGGVWAGGATDYNSALKSRLTITRDTSKSQVFLKMHSLQSEDTATYYCARDGGYSSSTLYAMDAWGQGTTVTVSA

SEQ ID NO: 3 - protein, 107 aa, Artificial Sequence
  synthetic peptide variable region of the light chain of Hu-alpha D11
DIQMTQSPSSLSASVGDRVTITCRASEDIYNALAWYQQKPGKAPKLLIYNTDTLHTGVPSRFSGSGSGTDYTLTISSLQPEDFATYFCQHYFHYPRTFGQGTKVEIK

SEQ ID NO: 4 - protein, 122 aa, Artificial Sequence
  synthetic peptide variable region of the heavy chain of Hu-alpha D11
EVQLVESGGGLVQPGGSLRLSCAASGFSLTNNNVNWVRQAPGKGLEWVGGVWAGGATDYNSALKSRFTISRDNSKNTAYLQMNSLRAEDTAVYYCARDGGYSSSTLYAMDAWGQGTLVTVSS

SEQ ID NO: 5 - protein, 452 aa, Artificial Sequence
  synthetic peptide humanised anti-NGF
EVQLVESGGGLVQPGGSLRLSCAASGFSLTNNNVNWVRQAPGKGLEWVGGVWAGGATDYNSALKSRFTISRDNSKNTAYLQMNSLRAEDTAVYYCARDGGYSSSTLYAMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 452 aa, Artificial Sequence
  synthetic peptide humanised Hu-alpha D11
EVQLVESGGGLVQPGGSLRLSCAASGFSLTNNNVNWVRQAPGKGLEWVGGVWAGGATDYNSALKSRFTISRDNSKNTAYLQMNSLRAEDTAVYYCARDGGYSSSTLYAMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 449 aa, Artificial Sequence
  synthetic peptide humanised Hu-alpha D11
EVQLVESGGGLVQPGGSLRLSCAASGFSLTNNNVNWVRQAPGKGLEWVGGVWAGGATDYNSALKSRFTISRDNSKNTAYLQMNSLRAEDTAVYYCARDGGYSSSTLYAMDAWGQGTLVTVSSASTKGPSVFPLAPSSKSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 214 aa, Artificial Sequence
  synthetic peptide Humanised Hu-alpha D11
DIQMTQSPSSLSASVGDRVTITCRASEDIYNALAWYQQKPGKAPKLLIYNTDTLHTGVPSRFSGSGSGTDYTLTISSLQPEDFATYFCQHYFHYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 9 aa, Homo sapiens
EVNINNSVF

SEQ ID NO: 10 - protein, 13 aa, Homo sapiens
GDKTTATDIKGKE

(19) United States
(12) Patent Application Publication
(10) Pub. No.: US 2018/0155415 A1
(43) Pub. Date: Jun. 7, 2018

(54) MOLECULES THAT ARE ABLE TO INHIBIT THE BINDING BETWEEN NGF AND THE TRKA RECEPTOR AS ANALGESICS WITH PROLONGED EFFECT

(71) Applicant: Flaminia Pavone, (US)

(72) Inventor: Flaminia Pavone, (US)

(21) Appl. No.: 15/367,546

(22) Filed: Dec. 2, 2016

(51) Int. Cl. C07K 16/22

(52) U.S. Cl. CPC C07K 16/22; C07K 2317/76; C07K 2317/565; A61K 2039/505; C07K 2317/622; C07K 2317/21; C07K 2317/24; C07K 2317/56

(57) ABSTRACT

Use of an anti-NGF antibody capable of inhibiting the binding between NGF and TrkA, capable of blocking the biological activity of TrkA for the preparation of a medicament for treating and/or preventing chronic pain.

### BACKGROUND TO THE INVENTION

The present invention relates to the use of molecules that are capable of inhibiting the binding between NGF and its receptor, TrkA. In particular, it relates to antibodies for one of the two molecules that, by blocking the biological activity of NGF, have a prolonged analgesic effect. Owing to the enduring analgesic effect thereof, they are an advantageous therapy for pathologies with persistent forms of pain, known also as chronic pain, such as but not limited to neuropathic or oncological pain.

### STATE OF THE ART

The nociceptive signals afferent to the spinal cord are carried by the fibres Aδ and C, the cell bodies of which (primary sensitive neurons) are located in the spinal dorsal ganglia (DRG). The primary sensitive neurons release glutamate together with ATP as an excitatory neurotransmitter, and various other substances such as substance P and CGRP (calcitonin-gene-related-peptide), (Hunt and Mantyh, 2001). The release of these excitatory neurotransmitters is controlled by various classes of receptors present on the afferent terminals, including those that are sensitive to capsaicin (vanilloid receptors, VR1), those activated by GABA, those activated by ATP itself and those activated by cannabinoids (CB1) (Sivilotti and Nistri, 1991; Hunt and Mantyh, 2001; Khakh, 2001; Morisset et al., 2001). One of the physiopathological mechanisms whereby chronic pain occurs is allodynia, i.e. the transformation of stimuli that are not normally painful into painful sensations. This phenomenon involves various ionic currents and thus various channels of the “ligand-gated” type, including the receptor for the capsaicin, VR1, and the ionotropic receptors for ATP (Khakh, 2001). The simultaneous activation of the receptors for VR1 and of those for ATP on spinal nociceptive interneurons generates a considerable accumulation of the excitatory synaptic signals with reinforcement of the transmission of the painful stimulus (Nakatsuka et al., 2002). On this basis, it is therefore clear that the ATP receptors (especially those belonging to the class P2X3) play a fundamental role in the pain paths (Burnstock, 2001). These receptors are present on the peripheral nerve terminals activated by algogenic stimuli, on the cell bodies of the neurons in the DRGs and on the presynaptic terminals thereof, as well as naturally on postsynaptic terminals in the spinal cord (Khakh, 2001). There is considerable evidence that shows that the system constituted by the nerve growth factor (NGF) and by the high-affinity receptor thereof TrkA (Levi-Montalcini, 1987; Levi-Montalcini et al., 1996; Frade and Barde, 1998; Kaplan, 1998) plays a fundamental role in the molecular processes underlying the main forms of “persistent” pain. This indicates a main therapeutical area (the one of pain, with particular reference to the “tonic” forms), for the antibodies that block the NGF/TrkA system (Levine, 1998). The development of sensitive nociceptive neurons depends greatly on NGF, and the responses of the adult nociceptors are modulated by the same factor (Julius and Basbaum, 2001). In particular, NGF exerts acute sensitisation of the capsaicin algogenic stimulus (Shu and Mendell, 1999). From a functional point of view, the nociceptive neurons, following chronic inflammation, develop alterations in the frequency and duration of their action potential. These phenomena regress by blocking endogenous NGF leading to a significant attenuation of the hyperexcitability that is typical of chronic painful states (Djouhri et al., 2001). NGF action in defining the pain threshold in adult nociceptors is mediated by the TrkA receptor, also through modulation of the response mediated by the VR1 receptor present on the nociceptive terminals. The TrkA-dependent potentiation of the VR1 response is thought to occur through the intracellular transduction pathway of the gamma version of phospholipase C (PLCgamma, Chuang et al., 2001). The levels of peripheral NGF are increased in inflammatory processes, while the administration of exogenous NGF has a hyperalgesic effect on rats and produces muscular pain in humans. Furthermore, NGF produces hypersensitisation to heat stimulation in humans and mammals in general. NGF is released by mastocytes, fibroblasts and other cell types in the peripheral sites where inflammatory processes occur. In particular the mastocytes appear to play a fundamental role (Woolf et al., 1996). As these cells produce NGF and at the same time express functional TrkA receptors on their surface (Nilsson et al., 1997), they are able to respond to NGF itself, in the presence of lysophosphatidylserine (Horigome et al., 1993; Kawamoto et al., 2002). As a result, the system NGF/TrkA appears to mediate mastocyte activation through a positive feedback autocrine mechanism allowing the local amplification of the algogenic inflammatory signal.

High levels of NGF are also found in neurons, where this neurotrophin is apparently responsible for the modifications of nerve fibres, associated with pain (Harpf et al., 2002). In certain forms of cancer, the excess of NGF facilitates the growth and infiltration of nerve fibres with induction of oncological pain (Zhu et al., 1999). Recent experimental studies show that, by blocking NGF, it could be possible to significantly reduce the formation of neuromas, responsible for neuropathic pain, without damaging the cell bodies of the lesioned neurons (Kryger et al., 2001). These results elicited significant interest in therapeutic approaches based on the reduction of the effects of NGF for the treatment of chronic pain (Saragovi and Gehring, 2000). In recent years, the involvement of the NGF/TrkA system in the molecular processes of pain transduction has also been demonstrated on a genetic base. In particular, mutations of the TrkA gene (localised on the chromosome 1q21-q22) are responsible for a hereditary recessive autosomic syndrome known as CIPA (“congenital insensitivity to pain with anhydrosis”), characterised by recurrent episodic fever, anhydrosis, absence of reaction to stimuli that cause pain, mental retardation and a tendency to self-mutilation (Indo et al., 1996; Saragovi and Gehring, 2000; Indo, 2001; Indo et al., 2001). Further confirmation of the involvement of NGF in the nociceptive response was recently obtained by the work on phenotype characterisation of anti-NGF transgenic mice (AD11). In these animals, the ectopic expression of the anti-NGF antibody αD11 produces a functional block of NGF in adult. This block translates in a consistent manner into an increase in the latency time of the response to harmful heat stimuli (Capsoni et al., 2000; Ruberti et al., 2000). Antibodies that are able to neutralise the biological activity of the NGF/TrkA system by blocking the ligand or the receptor may represent an important resource in pain therapy, in particular for persistent forms of pain. In this context, a very recent publication demonstrates that treatment with a neutralising anti-NGF antibody produces significant pain reduction in a murine oncological pain model (Sevcik et al., 2005). Nevertheless, in the administration protocol used by Sevcik et al., the maximum time lapse between the last injection of anti-NGF and observation of the behaviour does not exceed 4 days, so it is not a long-term effect.

A long-term effect can be defined as an effect which is still evident for at least 1-2 week(s), after the last administration of the antibody, implying that there is no necessary correlation between the effect and the bloodstream concentration of the antibody itself. A long-term effect may require new gene expression and may represent a permanent or prolonged modification of the original physiopathological state. In many cases, the drug that is capable of producing a long-term effect, may be defined as a “disease-modifying” active principle, i.e. capable of modifying in depth the course of the disease, unlike products displaying a simple pharmacological effect on the symptoms.

The authors of the present invention dispose of a panel of antibodies (directed against the NGF ligand) that are able to block the biological effects of NGF that are mediated by the TrkA ligand. Two reagents: αD11 (anti-NGF) and MNAC13 (anti-TrkA) are of particular interest. The comparison between the two antibodies, the one directed against the ligand and the other directed against the receptor, is of significant interest as the inhibition of the NGF ligand is not functionally equivalent to the inhibition of the TrkA receptor. Three points must in fact be considered:
i) stoichiometric reasons, in the context of the same system, the availability of ligand and of receptor may vary greatly and vary in a different fashion, over time;
ii) the presence of a second receptor for NGF (p75) that is shared by all the neurotrophins and mediates distinct biological functions with respect to TrkA (Hempstead, 2002);
iii) the presence, in nature, of “immature” forms of NGF (pre-pro-NGF), characterised by distinct properties, in terms of bioactivity, and binding preferably to the p75 receptor (Lee et al., 2001).

αD11 is a rat monoclonal antibody directed against mouse NGF (but also able to recognise rat and human NGF). Its interaction with NGF inhibits the binding thereof with TrkA, blocking the physiological action thereof (Cattaneo et al., 1988). αD11 also inhibits the binding of NGF to the p75 receptor. This anti-NGF antibody is absolutely unique in terms of its specificity of binding to its antigen (as compared to all the other neurotrophins), through the affinity of binding with the antigen (picomolar) and through neutralising features, showed both in vitro and in vivo (Cattaneo et al., 1988; Berardi et al., 1994; Molnar et al., 1997; Molnar et al., 1998). The alphaD11 epitope is located at the level of NGF loop I and/or NGF loop II that are exposed to the outer part of the molecule and spatially very close to each other. Moreover, the conserved reactivity of alphaD11 in different species is consistent with the epitope assignment, since amino acid residues of these two loops are highly conserved. The potent neutralising activity of alphaD11 shows that the recognised epitope is very close to the NGF receptor binding site. Moreover, the lack of cross reactivity of alphaD11 with other members of the neurotrophin family suggests that: i) the epitope is located in NGF regions that are not shared with other neurotrophins, ii) the epitope itself may be involved in the “specificity path” mediating NGF-TrkA recognition. The epitope recognized by the alphaD11 antibody on the NGF molecule was identified by testing the binding activity of the antibody towards an extensive panel of NGF mutants. On the basis of this systematic screening, a region (aa. 41-49, loop I) of the NGF molecule was identified that is highly expressed on top of NGF molecule and that is responsible (though not exclusively) for the binding of the antibody to its antigen (Gonfloni, 1995). As a matter of fact also the NGF aa. region 23-35 (loop II) may contribute to the binding.

The antibody MNAC13 is a mouse monoclonal antibody directed against the human TrkA receptor (Cattaneo et al., 1999; Pesavento et al., 2000), that is particularly effective in the inhibition of the process of TrkA activation by NGF and of downstream biological functions, both in vitro and in vivo (Cattaneo et al., 1999; Pesavento et al., 2000). The antibodies were characterized in detail from the point of view of the structure (Covaceuszach et al., 2001) and from the molecular interaction with the TrkA receptor (Covaceuszach et al., 2005).

On the basis of such in-depth knowledge around structure, by means of an innovative method, humanised versions of both αD11 and MNAC13 were generated (Hu-αD11 and Hu-MNAC13), displaying the same antigen binding features of the parental versions (patent application WO 05/061540).

The therapies that are currently available for treating pain of neuropathic origin (caused by a primary lesion or by a dysfunction of the nervous system, for example the pain associated by a lesion of the spinal cord), for treating oncological pain, and for numerous other forms of persistent pain (also of inflammatory nature) have been found to be of limited effectiveness. There is therefore an obvious need to identify and develop new molecules that have an analgesic activity and which work through a different action mechanism as compared to currently used analgesic drugs, in order to solve side effect related problems. The international patent application WO 02/20479 discloses small synthesis molecules that inhibit the TrkA receptor, having a potential analgesic activity. Nevertheless, the effect of these molecules on certain pain models has not been demonstrated. Furthermore, as compared to antibodies, the small molecules have the drawback of being more likely to penetrate the haemato encephalic barrier, with the possibility of serious side effects. In fact, the cholinergic neurons of the basal forebrain, a neuronal population that is affected by various forms of progressive neurodegeneration, including Alzheimer's disease (Saper et al., 1985), express the TrkA receptor and depend on NGF for correct functioning (Holtzman et al., 1992). The international patent application WO 01/78698 proposes the use of an NGF antagonist for preventing or treating chronic visceral pain, but not for neuropathic or oncological pain. Even if the application states that the antagonist can bind both NGF and the TrkA receptor, it is not demonstrated that, upon binding of the antagonist to the TrkA receptor, the receptor is functionally blocked. On the basis of the capacity of the two antibodies MNAC13 and αD11 to block the biological activity of NGF/TrkA, the two antibodies MNAC13 and αD11 and their respective humanised versions were tested in various (rodent) animal models of persistent pain, in particular in the model CCI (“Chronic Constriction Injury”, chronic constriction injury of the sciatic nerve), one of the models available for assessment of chronic pain of a neuropathic nature (Bennett and Xie, 1988).

### SUMMARY OF THE INVENTION

The object of the present invention is the use of an anti-NGF that is able to inhibit the binding between NGF and TrkA, for the preparation of a medicament for the treatment of chronic pain.

An anti-NGF molecule that blocks the biological activity of TrkA is defined as a molecule that acts as an antagonist in terms of the NGF binding with the TrkA receptor and comprises: synthetic molecule or monoclonal antibody or a biological/synthetic derivative thereof that:
i) binds to TrkA;
ii) inhibits the binding of NGF to the “native” TrkA receptor expressed on the surface of living cells (“native” meaning “in the natural in vivo conformation”); and
iii) blocks the biological activity that derives from the NGF binding with the same TrkA receptor.

The term “blocking the biological activity” does not simply mean blocking activation of the receptor, defined as blocking the conversion process of the receptor itself into an “active” state but also functional neutralisation of the biological consequences that are downstream of this activation process: second messengers, new gene expression, phenotypical and functional modifications. The molecule is not only able to block TrkA in a classic in vitro test (test of neuritic growth in PC12 cells), but also in vivo (functional block of the cholinergic neurons of the basal forebrain and block of the nociception in a classic “hot plate” test).

It is an object of the invention the use of an anti-NGF antibody capable of inhibiting the binding between NGF and TrkA for the preparation of a medicament for treating and/or preventing chronic pain. Preferably the antibody is able to recognise and bind to a NGF molecule domain containing the aa. 41-49 region of human or rat NGF: EVNINNSVF (SEQ ID No. 9), more preferably the domain contains also the aa 23-35 region: GDKTTATDIKGKE (SEQ ID No. 10). More preferably the antibody is capable of blocking the biological activity of TrkA.

There is also provided as an aspect of the invention a method of treatment and/or prevention of chronic pain in a subject comprising administering to the subject an effective amount of an anti-NGF antibody thereby to treat and/or prevent chronic pain in said subject. There is also provided a kit comprising a composition containing an anti-NGF antibody together with instructions directing administration of said composition to a subject in need of treatment and/or prevention of chronic pain thereby to treat and/or prevent chronic pain in said subject.

In a preferred aspect the variable region of the antibody light chain comprises at least one, more preferably two, most preferably three of the complementarity determining regions (CDRs) having the sequence selected from aa. 24 to aa. 34 of [symbol] ID No. 1; from aa. 50 to aa. 56 of SEQ ID No. 1; from aa. 89 to aa. 97 of SEQ ID No. 1.

In a further preferred aspect the variable region of the antibody light chain comprises essentially the sequence of SEQ ID No. 1.

```
(VL, SEQ ID No. 1):
                                              L CDR1 L CDR2
DIQMTQSPASLSASLGETVTIECRASEDIYNALAWYQQKPGKSPQLLIYNTDTLHTGVP
                                                          L CDR3
SRFSGSGSGTQYSLKINSLQSEDVASYFCQHYFHYPRTFGGGTKLELK
```

In a preferred aspect the variable region of the antibody heavy chain comprises at least one, more preferably two, most preferably three of the complementarity determining regions (CDRs) having the sequence selected from aa. 26 to aa. 35 of SEQ ID No. 2; from aa. 50 to aa. 65 of SEQ ID No. 2; from aa. 98 to aa. 111 of SEQ ID No. 2.

In a further preferred aspect the variable region of the antibody heavy chain comprises essentially the sequence of SEQ ID No. 2.

(VH, SEQ ID NO 2):
H CDR1 H CDR2
QVQLKESGPGLVQPSQTLSLTCTVSGFSLTNNNVNWVRQATGRGLEWMGGVWAGGATDY
H CDR 3
NSALKSRLTITRDTSKSQVFLKMHSLQSEDTATYYCARDGGYSSSTLYAMDAWGQGTTV
TVSA

The antibody may be in single chain form and comprises a light chain variable region and a heavy chain variable region joined by a linker.

Alternatively the antibody may comprise two light chains and two heavy chains.

In a preferred aspect of the invention the anti-NGF antibody is a human or humanised antibody. The skilled in the art shall select the proper humanisation method to design the antibody, a preferred method is the method as disclosed in WO 2005/061540.

Briefly, a “humanized” variant of the antibody variable region was obtained by grafting the Complementarity Determining Regions (CDRs) of the rat antibody on to a human immunoglobulin framework. The complete structural information obtained from X-ray diffraction studies concerning the Fab fragment of the αD11 antibody was exploited to select an acceptor framework of human origin. Two different criteria were adopted to minimize structural differences between the rat αD11 and the acceptor human antibody: i) level of primary structure homology, ii) level of tridimensional structure similarity. After choosing the framework, the replacement of human residues by rat counterparts was minimized to reduce the potential immunogenicity of the resulting humanized antibody.

Exemplary humanised antibodies comprise a light chain variable region which is a humanised derivative of SEQ ID No 1 (a rat origin sequence). Exemplary humanised antibodies comprise a heavy chain variable region which is a humanised derivative of SEQ ID No 2 (a rat origin sequence).

In a preferred aspect of the invention the variable region of the humanised antibody light chain comprises essentially the sequence of SEQ ID No. 3.

```
SEQ ID NO 3 (VL, variable region of the light chain of Hu-αD11):
                                                      L CDR1 L CDR2
DIQMTQSP [symbol] SLSAS [symbol] G [symbol] VTI [symbol] CRASEDIYNALAWYQQKPGK [symbol] P [symbol] LLIYNTDTLHTGVP
                                                            L CDR3
SRFSGSGSGT [symbol] Y [symbol] L [symbol] I [symbol] SLQ [symbol] ED [symbol] A [symbol] YFCQHYFHYPRTFG [symbol] GTK [symbol] E [symbol] K
```

In a preferred aspect of the invention the variable region of the humanised antibody heavy chain comprises essentially the sequence of SEQ ID No. 4.

```
SEQ ID No. 4 (VH, variable region of the heavy chain of Hu-αD11):
                                                    H CDR1 H CDR2 [symbol]
VQL [symbol] ESG [symbol] GLVQP [symbol] L [symbol] L [symbol] C [symbol] SGFSLTATNNVNWVRQA [symbol] G [symbol] GLEW [symbol] GGVWAGGATDY
                                                                                    H CDR3
NSALKSR [symbol] TI [symbol] RD [symbol] SK [symbol] L [symbol] M [symbol] SL [symbol] EDTA [symbol] YYCARDGGYSSSTLYAMDAWGQGT [symbol] V
```

TVSS

The above described humanized variable regions were cloned into appropriate expression vectors into a human IgG1 or IgG4 isotype format and transfected into mammalian cell lines to allow for expression, purification and pharmacological characterization.

Different variants of Hu-αD11 (complete IgG: heavy chain+light chain) were finally produced (differing because of different constant parts).

In a preferred aspect of the invention the humanised antibody light chain has essentially the sequence of SEQ ID No. 8.

SEQ ID NO 8, Hu-αD11 Vk human Ck
DIQMTQSP [symbol] SLSAS [symbol] G [symbol] VTI [symbol] CRASEDIYNALAWYQQKPGK [symbol] P [symbol] LLIYNTDTLHTGVP
SRFSGSGSGT [symbol] Y [symbol] L [symbol] I [symbol] SLQ [symbol] ED [symbol] A [symbol] YFCQHYFHYPRTFG [symbol] GTK [symbol] E [symbol] KRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC

(Italics = variable regions; Bold = mutations in the rat sequence in the humanization process; Underlined = CDRs)

In a preferred embodiment, the humanised anti-NGF heavy chain has essentially one of the following 3 sequences:

SEQ ID NO 5, Hu-antiNGF (VH) human IgG1 [symbol]
VQL [symbol] ESG [symbol] GLVQP [symbol] L [symbol] L [symbol] CS [symbol] SGFSLTNNNVNWVRQA [symbol] G [symbol] GLEW [symbol] GGVWAGGATDY
NSALKSR [symbol] TI [symbol] RD [symbol] SK [symbol] L [symbol] M [symbol] SL [symbol] EDTA [symbol] YYCARDGGYSSSTLYAMDAWGQGT [symbol] V
TVS [symbol] ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO 6, αHu-D11 (VH) human IgG1* (IgG1 with N297A mutation,
as described by Bolt et al., 1993) [symbol]
VQL [symbol] ESG [symbol] GLVQP [symbol] L [symbol] L [symbol] C [symbol] SGFSLTNNNVNWVRQA [symbol] G [symbol] GLEW [symbol] GGVWAGGATDY
NSALKSR [symbol] TI [symbol] RD [symbol] SK [symbol] L [symbol] M [symbol] SL [symbol] EDTA [symbol] YYCARDGGYSSSTLYAMDAWGQGT [symbol] V
TVS [symbol] ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKTK
PREEQYASTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO 7, Hu-αD11 (VH) human IgG4 [symbol]
VQL [symbol] ESG [symbol] GLVQP [symbol] L [symbol] L [symbol] C [symbol] SGFSLTNNNVNWVRQA [symbol] G [symbol] GLEW [symbol] GGVWAGGATDY
NSALKSR [symbol] TI [symbol] RD [symbol] SK [symbol] L [symbol] M [symbol] SL [symbol] EDTA [symbol] YYCARDGGYSSSTLYAMDAWGQGT [symbol] V
TVS [symbol] ASTKGPSVFPLAPSSKSTSESTAALGCLVKDYFPEPVTVSWNSGALTSGVHTFPA
VLQSSGLYSLSSVVTVPSSSLGTKTYTCNVDHKPSNTKVDKRVESKYGPPCPSCPAPEF
LGGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK

(Italics = variable regions; Bold-mutations in the rat sequence in the humanization process; Underlined = CDRs; N297A mutation to abolish glycosilation site).

In a preferred aspect the molecules of the invention are used for the preparation of a medicament for the pain of the chronic inflammatory type, preferably caused by pancreatitis, kidney stones, headaches, dysmenorrhoea, musculoskeletal pain, sprains, visceral pain, ovarian cysts, prostatitis, cystitis, interstitial cystitis, post-operative pain, migraine, trigeminal neuralgia, pain from burns and/or wounds, pain associated with trauma, neuropathic pain, pain associated with musculoskeletal diseases, rheumatoid arthritis, osteoarthritis, ankylosing spondilitis, periarticular pathologies, oncological pain, pain from bone metastases, pain from HIV.

Alternatively the pain is a neuropathic pain or an oncological pain.

According to International Association for the Study of Pain (IASP, www.iasp-pain.org <http://www.iasp-pain.org/>), pain is generally defined as “An unpleasant sensory and emotional experience associated with actual or potential tissue damage, or described in terms of such damage or both”. The essential element in all forms of pain is the activation of specialized high-threshold receptors and nerve fibers to warn the organism of potential tissue damage. The involvement of inflammatory cells and processes is a common element in many pain states. The term “acute pain” means immediate, generally high threshold, pain brought about by injury such as a cut, crush, burn, or by chemical stimulation. The term “chronic pain,” as used herein, means pain other than acute pain. It is understood that chronic pain often is of relatively long duration, for example, months or years and can be continuous or intermittent.

The anti-NGF antibody is suitably administered systemically. Systemic administration of the anti-NGF antibody can be performed by injection, e.g. continuous intravenous infusion, bolus intravenous infusion, subcutaneous or intramuscular injection. Alternatively other fauns of administration (e.g. oral, mucosal, via inhalation, sublingually, etc.) may also be used. Local delivery of the antibody can be performed by local administration eg intra-articular injection or subcutaneous, intramuscular injection in the vicinity of affected tissues.

The anti-NGF antibody will suitably be formulated in a pharmaceutical composition appropriate for the intended route of administration. Solutions for injection will suitably contain the antibody dissolved or dispersed in an aqueous medium (eg water for injection) containing appropriate buffers and molarity modifiers eg phosphate, salt and/or dextrose.

Treatment regimen i.e. dose, timing and repetition, can be represented by single or repeated administrations (eg injections) of the product by the chosen administration route. The interval of dose administration can be subject to modifications depending on the extent and duration of the clinical response, as well as the particular individual and the individual clinical history. Suitably the anti-NGF antibody has a long duration of action. In particular the clinical effect of the antibody extends following administration as long as 21 days as determined from animal studies. Furthermore, preliminary data imply that anti-NGF antibodies may manifest clinical benefit for a longer period than that in which its presence can be detected in a relevant biological matrix such as serum or plasma following its administration.

In light of the intended long duration of action (i.e. an effect suitably lasting at least one week, or preferably at least two weeks eg at least three weeks or at least four weeks), suitably the antibody may be administered to subjects at a frequency of not more than once per week eg not more than once per two weeks or once per three weeks or once per four weeks.

A suitable dose of the anti-NGF antibody will typically range from 0.1 mg/kg to 10 mg/kg body weight

Novel antibodies and compositions containing them disclosed herein are claimed as an aspect of the invention.

Non-limitative embodiments of the present invention will now be disclosed, with particular reference to the following figures:

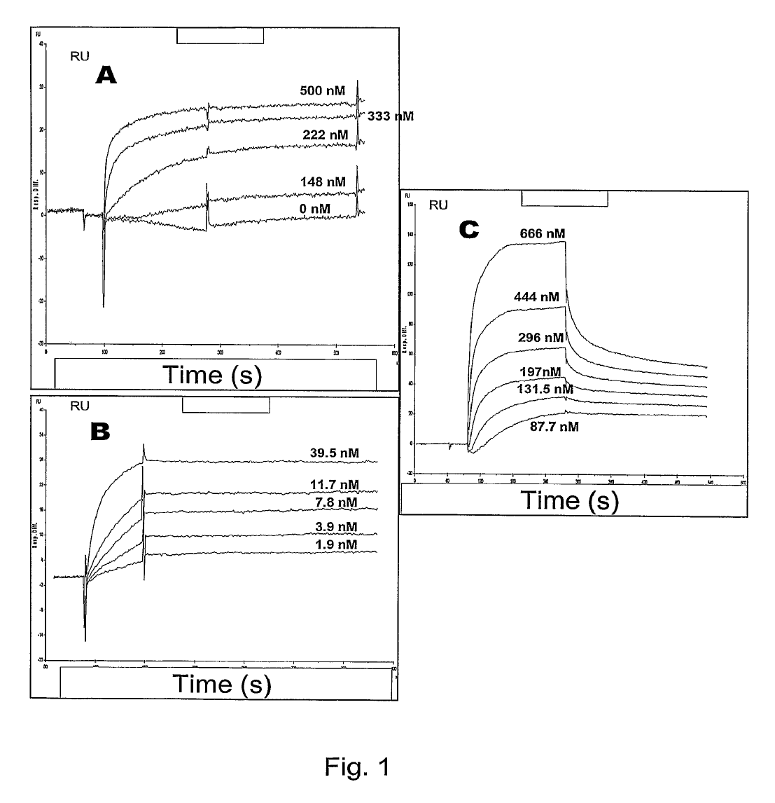

FIG. 1: BIAcore analysis of the binding of the αD11 anti-NGF antibody to mouse NGF (m-NGF) and recombinant mouse proNGF (rm-proNGF). The αD11 anti-NGF antibody was immobilized on flow cell 2, while flow cell 1 was left as a blank. Each curve is obtained subtracting the background signal (measured in cell 1) to the signal measured in cell 2. The Surface Plasmon Resonance signal gives the amount of surface-bound component at each stage and is expressed in resonance units (RU).

For m-NGF binding, the immobilization of the antibody was of 3000 resonance units (RU) in the experiment of panel A and of 6000 RU in the experiment of panel B. The injected concentrations of the m-NGF are indicated on top of each curve. From a complete analysis of the data, the affinity parameters were evaluated, and resulted to be the following: KA=3.55·10¹¹ 1/M; KD=2.81·10⁻¹² M (chi² value of 0.123).

For rm-proNGF binding (panel C), the immobilization of the antibody was of 3000 RU. The injected concentrations of the rm-proNGF are indicated on top of each curve. A kinetic analysis of the data allowed to evaluate the following parameters: KA=1.2·10⁹ 1/M; KD=1.9·10⁻⁹ M (chi² value of 0.09).

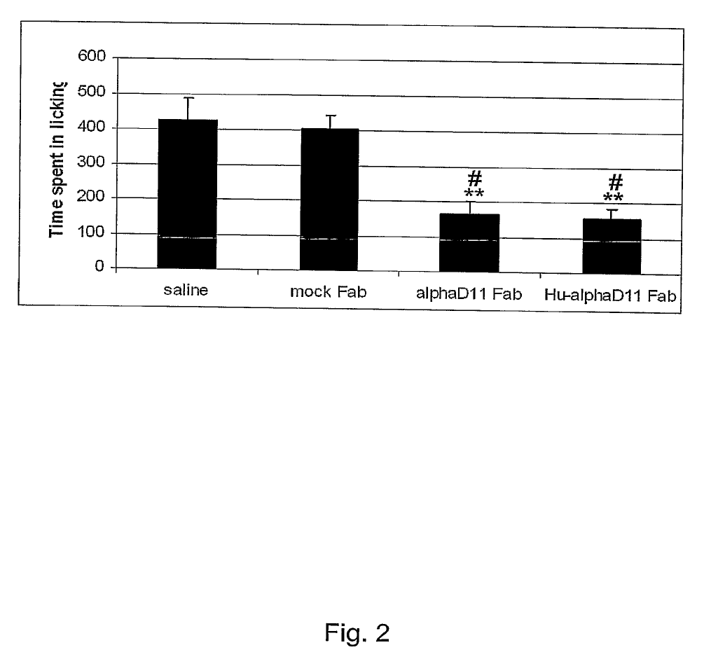

FIG. 2: Effect of Fab aD11 (alphaD11) and Fab Hu-aD11 (Hu-alphaD11) anti-NGF antibodies on the formalin-evoked pain (phase 2 of the formalin test: 15-40 min. Phase 2 corresponds to inflammation-related pain). Mice were subcutaneously injected with 5% formalin in the dorsal portion of the right hindpaw.

Treatment consisted in antibody injection (Fab alphaD11 or Fab Hu-alphaD11 vs either mock Fab or saline) performed (in the same paw as for formalin) 45 min, before formalin injection and testing (single dose of each antibody: 12.5 μg). Each experimental group included at least 8 animals. Statistical analysis of data showed a significant analgesic effect of anti-NGF treatment (both for the parental and the humanized version of the antibody) that was clearly specific for the second phase (inflammatory) of pain response (time spent in licking): The effect of anti-NGF antibodies (both parental and humanized versions) is statistically different (ANOVA) with respect to either saline (**p<0.01) or mock Fab treatment (#p<0.05).

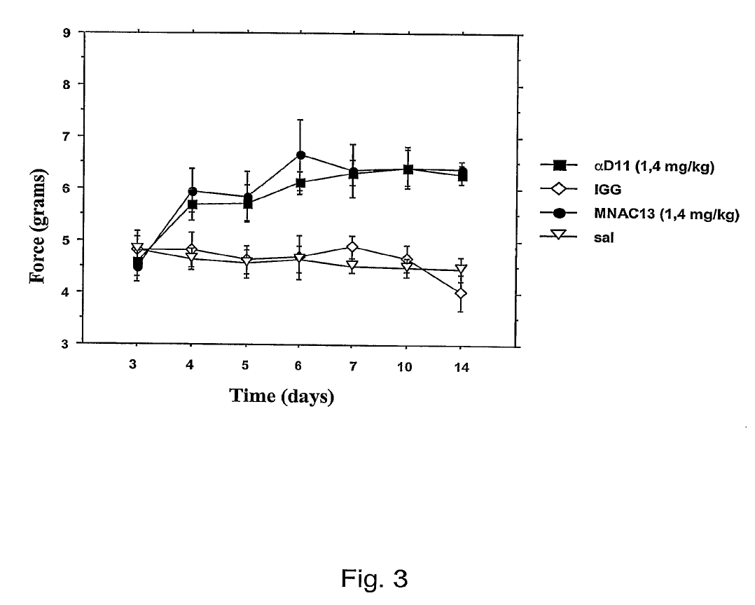

FIG. 3: Effect of the anti-TrkA monoclonal antibody MNAC13 (1.4 mg/kg) and anti-NGF monoclonal antibody αD11 (1.4 mg/kg) on neuropathic pain: mechanical allodynia measured by means of a plantar dynamic aesthesiometer; CD1 mice subjected to chronic constriction of the sciatic nerve; the antibodies are injected I.P. at days 3, 4, 5, 6 after lesion of the sciatic nerve. Observation period: from day 3 to day 14. As a negative control, both saline (sal) and mouse immunoglobulins (IgG, 1.4 mg/kg) were used. Results were expressed in terms of absolute value (grams) of the threshold force for the hindpaw ipsilateral to lesion. The values were subjected to statistical analysis by means of an analysis of the variance (ANOVA) for repeated measurements, in which both the “treatment” factor and the repeated measurement (days) were significant with p<0.01. The animals treated with anti-TrkA or anti-NGF are significantly different from the controls from day 4 to day 14.

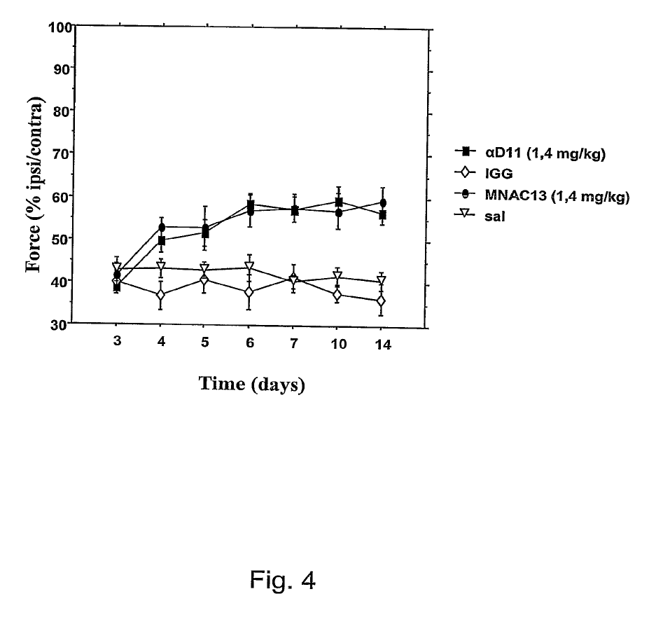

FIG. 4: Effect of the anti-TrkA monoclonal antibody MNAC13 (1.4 mg/kg) and anti-NGF αD11 antibody (1.4 mg/kg) on neuropathic pain: mechanical allodynia measured by means of a plantar dynamic aesthesiometer; CD1 mice subjected to chronic constriction of the sciatic nerve; the antibodies were injected I.P. at days 3, 4, 5, 6 after lesion of the sciatic nerve. Observation period: from day 3 to day 14. As a control, both saline (sal) and mouse immunoglobulins (IgG, 1.4 mg/kg) were used. Results were expressed as a percentage, % (ratio between the threshold force of the hindpaw ipsilateral to lesion and that corresponding to the contralateral hindpaw). The corresponding absolute values were subjected to statistical analysis by means of an analysis of the variance (ANOVA) for repeated measurements, in which both the “treatment” factor and the repeated measurement (days) were significant with p<0.01 (at least). The animals treated with anti-TrkA o anti-NGF were significantly different from the controls from day 4 to day 14.

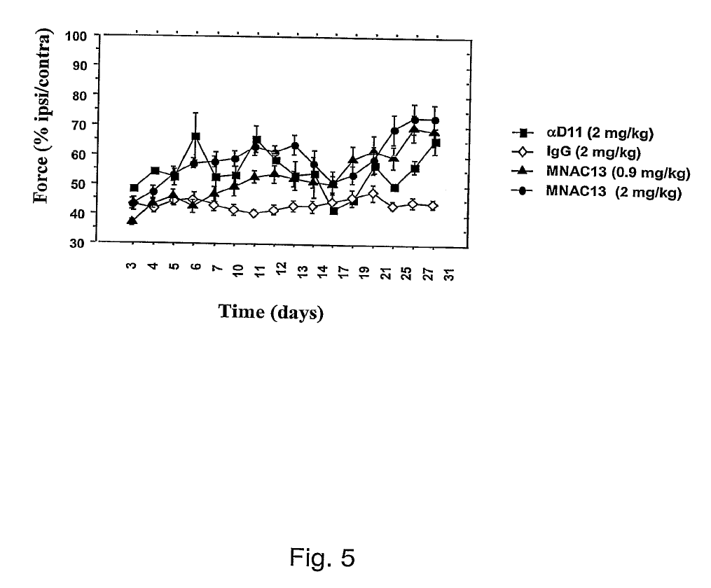

FIG. 5: Comparison between the effects of the anti-TrkA monoclonal antibody MNAC13 (2 doses: 0.9 and 2 mg/kg) and those of the anti-NGF monoclonal antibody αD11 (2 mg/kg dose), on neuropathic pain: mechanical allodynia measured by means of a plantar dynamic aesthesiometer; CD1 mice subjected to chronic constriction of the sciatic nerve; the antibodies were injected I.P. at days 3, 4, 5, 6, 7, 8, 9, 10 after lesion of the sciatic nerve. Observation period: from day 3 to day 31. As a negative control, mouse immunoglobulins were used (IgG, 2 mg/kg). Results were expressed as a percentage % (ratio between the threshold force of the hindpaw ipsilateral to lesion and that corresponding to the contralateral hindpaw). The corresponding absolute values were subjected to statistical analysis by means of an analysis of the variance (ANOVA) for repeated measurements, in which both the “treatment” factor and the repeated measurement (days) were significant with p<0.01 (at least). The animals treated with MNAC13 were significantly different from the controls up to the last day of observation (31), from day 5 (greater dose of MNAC13) or from day 7 (lesser dose of MNAC13). The animals treated with αD11 were significantly different from the controls up to the last day of observation (31), from day 4 to day 14, and from day 21 to day 31.

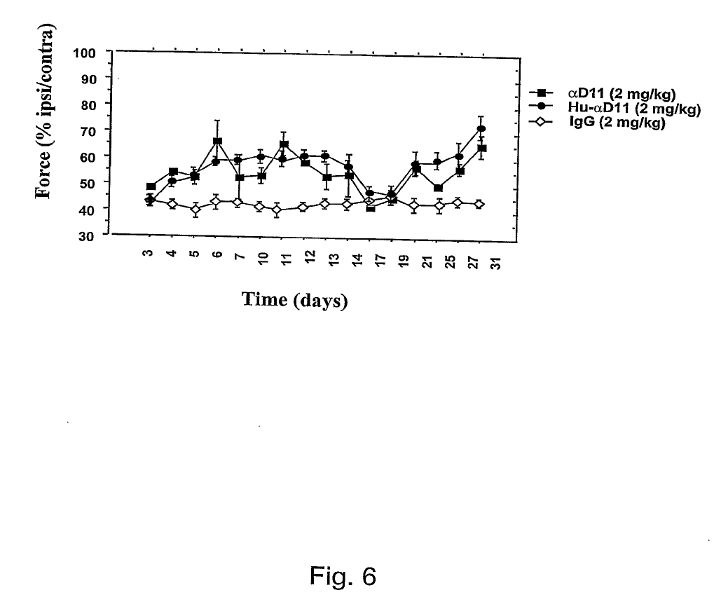

FIG. 6: Comparison between the effects of the parental (αD11) and humanized (Hu-αD11, human IgG4 format) version of the anti-NGF neutralizing antibody (1 dose tested: 2 mg/Kg) on neuropathic pain: mechano-allodynia measured by means of dynamic plantar aestesiometer; CD1 mice subject to CCI (chronic constriction injury) of the sciatic nerve; antibodies I.P. injected at days 3, 4, 5, 6, 7, 8, 9, 10, following lesion of the sciatic nerve. Observation period: from day 3 to day 31. Rat Immunoglobulins were employed for negative control (IgG, 2 mg/kg). Results expressed as % (ratio between the threshold force for the hindpaw ipsilateral to lesion and the contralateral hindpaw). Analysis of variance (ANOVA) for repeated measures on corresponding absolute values in which both the factor “treatment” and the repeated measure (days) were significant with (at least) p<0.01. Animals treated with either αD11 or Hu-αD11 were significantly different from controls up to the last observation day (31), from day 4 to day 14, as well as from day 21 up to day 31.

### METHODS

Production of Monoclonal Antibodies

The monoclonal antibodies MNAC13 and αD11 are produced from a hybridoma supernatant, according to standard methods, disclosed above (Galfre and Milstein, 1981; Cattaneo et al., 1988; Cattaneo et al., 1999). The supernatant containing each antibody was subjected to precipitation (29% ammonium sulphate), followed by dialysis against PBS 1× (Spectra-Por 12/14K membrane, Spectrum) and affinity chromatography on sepharose protein G column (4-Fast Flow, Amersham Biosciences). Elution occurred by means of a low pH (HCl 5 mM) solution that was neutralised upon collection. The final eluate was concentrated (Amicon Ultra-15, 50K, Millipore) to obtain preparations of purified antibody in concentrations between 1 and 5 mg/ml.

The Fab (Fragments Antigen binding) version of the αD11 antibody was produced as previously described (patent application WO 05/061540, Covaceuszach et al., 2004). Briefly, Fab fragments were obtained from the correspondent whole monoclonal antibodies (IgG format) by papain proteolysis, followed by a ion exchange chromatography purification step and concentration of the Fab fragments collected in the flow-through. In order to separate the Fab fragments from the quite low amount of uncleaved IgG that was still present, size exclusion chromatography on a Superdex G75 column (Pharmacia) was performed using an FPLC system (Pharmacia), followed by a final concentration step.

As far as the humanised versions (IgG1/IgG1*/IgG4) of the 2 antibodies (Hu-αD11 and Hu-MNAC13) are concerned, they were also purified as disclosed above, starting from the supernatants of stably transfected cell lines, which were stable cotransfectants for the heavy chain (pVH/CMVexpress) and the light chain (pVL/CMVexpress) of each antibody. The vectors used have been disclosed previously (patent application WO 05/061540). The stable co-transfected clones were obtained through double selection with G418 and mycophenolic acid. In order to produce the IgG4 variant of Hu-αD11, since the pVH/CMVexpress vector comprises the constant part of human IgG1, this was replaced by the corresponding Fc region of IgG4 (cloned by RT-PCR from human lymphocyte RNA). The IgG1* variant (=IgG1 with the N297A mutation described by Bolt et al., 1993) was generated by site directed mutagenesis.

Surface Plasmon Resonance Studies

Experiments were performed on CMS5 chips with amine coupling, using a BIAcore 2000 machine. Coupling was performed with a specific kit purchased at BIAcore and the coupling reaction was carried out according to manufacturer's instructions.

Anti-NGF antibodies were immobilized on chip, while mouse NGF (m-NGF, Alomone) or recombinant mouse proNGF (rm-proNGF) were injected at decreasing concentrations to obtain binding curves.

The flow used in the experiments was of 30 μl/min, unless otherwise indicated. The regeneration of the chip was carried out in all cases with a pulse (10 μL) of 10 mM Glycine pH 1.5. Data collected were analyzed using the Package BIAevaluation 3.0.

The apparent equilibrium constant K_{D} is defined as the kₐ/k_{d} ratio.

Experiments in Murine Pain Models

The animals were treated and handled in accordance with the guidelines of the IASP Ethical Committee and the Italian national law (DL116/92, application of European Direction 86/609/EEC) on the use of animals in research. Every necessary effort was made to minimise the suffering of the animals and to use the minimum amount of animals required to produce reliable scientific data.

Formalin Test

For the preliminary formalin tests (Porro and Cavazzuti, 1993), CD1 male mice (Charles River Labs, Como, Italy) were used, weighing 35-40 g at the beginning of the experiments. Upon their arrival in the laboratory (at least 2 weeks before the experiments), mice were housed in standard transparent plastic cage (4 for cage) at constant temperature (22+1° C.) and relative humidity (60%), under a regular light/dark schedule (light 7.00-19.00). Food and water were unlimited. The experiments were carried out between 09.00 and 14.00 hours. For the formalin test, one animal at a time was placed in a transparent plexiglass cage (30×12×13 cm), and allowed to move freely for 30 min before the beginning of the test. After this adaptation period, 20 μl of formalin solution (5% in saline) were subcutaneously (sc) injected into the dorsal surface of the right hind paw of mice using a microsyringe equipped with a 26-gauge needle and the observation period started. A mirror was placed behind the cage and a videocamera in front of the cage to allow an unimpeded view of the animal's hind-paws. The licking activity, i.e. the total amount of time the animal spent licking and/or biting the injected paw, was taken as index of pain. The licking activity was recorded continuously for 40 min and calculated in blocks of consecutive 5-min periods (phase 2 corresponds to the block 15-40 min and can be identified with inflammation-related pain). In addition, to assess the effects of formalin injection on the spontaneous behaviours, general activity (time spent exploring the environment during walking, rearing and leaning), and self-grooming (time spent for face and body cleaning) during the formalin test were also continuously recorded for 40 min. No significant differences following treatment with anti-NGF antibodies were observed for these parameters. In this set of experiments, antibodies were administered as Fabs (fragments antigen binding; single dose of each antibody: 12.5 μg per animal).

Each mouse was subcutaneously (sc) injected 45 min before the test with anti-NGF antibody (either parental or humanized) or irrelevant Fab into the dorsal surface of the right hind paw using a Hamilton micro-syringe with a 26-gauge needle (volume injected=20 μl). Each animal underwent only one treatment. Testing was performed blind as for treatment group to which each subject belonged. The two phases characterizing the formalin test were separately analysed by one-way ANOVAs.

Sciatic Nerve Surgery

Male CD1 mice, weighing approximately 35 g, were anaesthetised (intraperitoneal injection with 500 mg/kg chloral hydrate), the sciatic nerve of the right hind leg was exposed to undergo loose ligature by means of stitching thread according to the chronic constriction lesion model (CCI) of the sciatic nerve, disclosed by Bennett and Xie (1988). The loose ligature of the sciatic nerve, at level of the upper thigh, induced peripheral mononeuropathy characterised by thermal/mechanical allodynia and hyperalgesia. By ligation of the nerve at 3 different but near points, the neuropathy was fully developed 3 days following the lesion and lasted for 2-3 months.

Pharmacological Treatment

Starting from the third day following the lesion, anti-NGF (αD11) blocking antibodies or anti-TrkA (MNAC13) antibodies were administered in an entire form (Mab) that were diluted in saline solution (vehicle), as indicated in Table I. As controls, mouse or rat irrelevant immunoglobulins used (IgG), in the same dose as the blocking antibodies (at the greater dose if 2 doses were used), or saline solution. Each experimental group included N=10 animals (unless explicitly stated otherwise).

TABLE I
Administration protocols and measurement
of mechanical allodynia.

| | | | Allodynia |
|---|---|---|---|
| Antibody | Dose | Administration i.p. | measurement |
| MNAC 13 | 50 μg/mouse = | 4, at days 3, 4, 5, 6 after | Days 3 to 14 |
| αD11 | 1.4 mg/kg | lesion | |
| MNAC 13 | 70 μg/mouse = | 8, at days 3, 4, 5, 6, 7, 8, | Days 3 to 31 |
| αD11 | 2 mg/kg | 9, 10 after lesion | |
| MNAC 13 | 30 μg/mouse = | | |
| | 0.9 mg/kg | | |

Mechanical allodynia was measured by means of a plantar dynamic aesthesiometer (Ugo Basile), as indicated in Table I. Day 3 was considered the baseline. The same protocols were used to assess the analgesic action of the humanised versions of the two antibodies MNAC13 and αD11.

Statistical Analysis of Results (CCI Experiments)

The results were expressed in 2 different ways, both as an absolute value of the threshold force value (in grams) that was sufficient for the animal to retract the hind leg that is ipsilateral to the lesion, or in percentage value, as the ratio between the absolute values of the hind legs (ipsilateral/contralateral). The values were subjected to statistical analysis by means of an analysis of the variance (ANOVA) for repeated measurements, in which both the “treatment” factor and the repeated measurement (days) were significant with p<0.01.

Results

Binding

A BIACORE study was performed, aimed at further characterising the binding properties of the αD11 anti-NGF antibody (and its humanized variant) by evaluating the binding affinity of this antibody for mouse NGF and recombinant mouse pro-NGF. FIG. 1 shows the results of these experiments: the αD11 antibody binds with different kinetics on NGF and proNGF. Similar results were also obtained with Hu-αD11.

The very small dissociation constant from NGF is representative of a very tight binding of the antibody to its antigen, and is quite a unique example among antibodies binding kinetics. By comparing the anti-NGF antibody binding to NGF and to proNGF, it is possible to assess that, in the latter case, the affinity is almost three orders of magnitude lower (nanomolar instead of picomolar). Considering that proNGF differs from NGF only by a short stretch of additional amino acids, this difference in binding affinities is absolutely unexpected and surprising.

As proNGF preferably binds to p75 (Lee, 2001), whereas mature NGF has a higher affinity for the TrkA receptor, αD11 and HuαD11 can be considered as novel selective inhibitors of the TrkA-mediated pathway, a remarkable property that has a particular relevance in view of the clinical use of anti-NGF neutralizing antibodies.

Inflammatory Pain

A first set of in vivo experiments, performed in mice and concerning formalin-evoked pain (inflammatory pain), demonstrated that:

(i) the αD11 anti-NGF antibody (in Fab format) was able to significantly reduce the pain response (formalin test: phase 2), as compared to an irrelevant Fab;

(ii) the same result could be obtained by replacing αD11 with its humanized variant (Hu-αD11, FIG. 2).

This means that Hu-αD11 displays as powerful analgesic properties as αD11 in a relevant model of inflammatory pain.

Neuropathic Pain

The results on the CCI model showed that the two blocking antibodies MNAC13 and αD11 (FIG. 3 and FIG. 4) had a significant analgesic effect. In particular, a similar result was observed for the two antibodies at the 1.4 mg/kg dose. As shown in FIG. 3 and FIG. 4, they started to have an analgesic effect from the second day of administration (day 4), reaching the maximum effect around day 6, keeping substantially the same analgesic efficacy for the entire duration of the observation until day 14. Expressing the result in percentage terms (ratio between the threshold force of the hindpaw ipsilateral to lesion and that corresponding to the contralateral hindpaw), as in FIG. 4, it can be stated that for each of the two blocking antibodies, the maximum percentage value was around 60%, being around 40% for the control groups (IgG and saline).

When the animals were observed for 4 weeks, up to day 31, administration of the antibodies blocking the NGF-TrkA system (FIG. 5 and FIG. 6) revealed a two-phase effect. The first phase of analgesic efficacy (from day 3 to day 17, i.e. until a week after the last injection) was characterised by a maximum effect around days 11-12. After a reduction of the effect (up to day 17), a second analgesic phase was observed with an increase in the effect up to day 31. Two phases in the analgesic action of NGF/TrkA blocking antibodies can therefore be distinguished: the first (“pharmacological” effect), that comprises the treatment period and the first week after the last injection of antibody (the week during which the effect diminishes, parallel to the haematic concentration of the antibody); the second, which identifies a long-term effect, probably requiring new genic expression, which is an effect that gives these antibodies the unique feature (in the field of neuropathic pain) of being a “disease-modifying” active principle, i.e. capable of modifying in depth the course of the disease, unlike the products currently used in this therapeutical context, which demonstrate a simple pharmacological effect on the symptoms. In FIG. 5, the analgesic effect of the 2 doses of MNAC13 anti-TrkA (2 and 0.9 mg/Kg) was compared with that of αD11 (2 mg/kg). The results are expressed in percentage terms. The temporal profile of αD11 efficacy is similar to that of MNAC13, although, at day 17, the animals treated with αD11 were indistinguishable from the controls (IgG), whilst all those treated with MNAC13 still differed significantly (p<0.01). From day 21 αD11 recovered analgesic effect, which reached a final level (day 31) that was similar to that of MNAC13 (greater than 60%, as compared to 40% of controls).

Substantially identical results to those illustrated above were obtained when instead of the αD11 antibody, the variants of its humanised version (Hu-αD11) were employed (dose used: 2 mg/kg for each antibody), confirming that the latter have the same analgesic properties as the parental version. The antibody was humanised with the method of WO2005/061540, both at the light (SEQ ID No. 3) and the heavy chain (SEQ ID No. 4) variable regions. To construct whole humanised antibodies, different constant regions were utilised, as above described (SEQ ID No. 5-8).

As typical example of the equivalence in terms of analgesic activity (CCI) of parental and humanized antibodies, FIG. 6 shows the comparison between αD11 and Hu-αD11 (IgG4 format).

On this basis, it is possible to state that Hu-αD11 has the same long-term effect as its parental version.

### BIBLIOGRAPHY

Bennett G J, Xie Y K (1988). Pain 33:87-107.

Berardi N, Cellerino A, Domenici L, Fagiolini M, Pizzorusso T, Cattaneo A, Maffei L (1994) Proc Natl Acad Sci USA 91:684-688.

Bolt S, Routledge E, Lloyd I, Chatenoud L, Pope H, Gorman S D, Clark M, Waldmann H (1993) Eur J Immunol 23: 403-411.

Burnstock G (2001) Trends Pharrnacol Sci 22:182-188.

Capsoni S, Ugolini G, Comparini A, Ruberti F, Berardi N, Cattaneo A (2000) Proc Natl Acad Sci USA 97:6826-6831.

Cattaneo A, Rapposelli B, Calissano P (1988) J Neurochem 50:1003-1010.

Cattaneo A, Capsoni S, Margotti E, Righi M, Kontsekova E, Pavlik P, Filipcik P, Novak M (1999) J Neurosci 19:9687-9697.

Chuang H H, Prescott E D, Kong H, Shields S, Jordt S E, Basbaum A I, Chao M V, Julius D (2001) Nature 411:957-962.

Covaceuszach S, Cattaneo A, Lamba D (2001) Acta Crystallogr D Biol Crystallogr 57:1307-1309.

Covaceuszach S, Cassetta, A., Cattaneo A, Lamb a D (2004) Acta Crystallogr D Biol Crystallogr 60:1323-1327.

Covaceuszach S, Cattaneo A, Lamba D (2005) Proteins 58:717-727.

Djouhri L, Dawbarn D, Robertson A, Newton R, Lawson S N (2001) J Neurosci 21:8722-8733.

Frade J M, Barde Y A (1998) Bioessays 20:137-145.

Galfre G, Milstein C (1981) Methods Enzymol 73:3-46.

Gonfloni S (1995) Recombinant antibodies as structural probes for neurotrophins. SISSA PhD Thesis.

Harpf C, Dabernig J, Humpel C (2002) Muscle Nerve 25:612-615.

Hempstead B L (2002) Curr Opin Neurobiol 12:260-267.

Holtzman D M, Li Y, Parada L F, Kinsman S, Chen C K, Valletta J S, Zhou J, Long J B, Mobley W C (1992) Neuron 9:465-478.

Horigome K, Pryor J C, Bullock E D, Johnson E M, Jr. (1993) J Biol Chem 268:14881-14887.

Hunt S P, Mantyh P W (2001) Nat Rev Neurosci 2:83-91.

Indo Y (2001) Hum Mutat 18:462-471.

Indo Y, Tsuruta M, Hayashida Y, Karim M A, Ohta K, Kawano T, Mitsubuchi H, Tonoki H, Awaya Y, Matsuda I (1996) Nat Genet 13:485-488.

Indo Y, Mardy S, Miura Y, Moosa A, Ismail E A, Toscano E, Andria G, Pavone V, Brown D L, Brooks A, Endo F, Matsuda I (2001) Hum Mutat 18:308-318.

Julius D, Basbaum A I (2001) Nature 413:203-210.

Kaplan D R (1998) Prog Brain Res 117:35-46.

Kawamoto K, Aoki J, Tanaka A, Itakura A, Hosono H, Arai H, Kiso Y, Matsuda H (2002) J Immunol 168:6412-6419.

Khakh B S (2001) Nat Rev Neurosci 2:165-174.

Kryger G S, Kryger Z, Zhang F, Shelton D L, Lineaweaver W C, Buncke H J (2001) J Hand Surg [Am] 26:635-644.

Lee R, Kermani P, Teng K K, Hempstead B L (2001) Science 294:1945-1948.

Levi-Montalcini R (1987) Science 237:1154-1162.

Levi-Montalcini R, Skaper S D, Dal Toso R, Petrelli L, Leon A (1996) Trends Neurosci 19:514-520.

Levine J D (1998) Neuron 20:649-654.

Molnar M, Ruberti F, Cozzari C, Domenici L, Cattaneo A (1997) Neuroreport 8:575-579.

Molnar M, Tongiorgi E, Avignone E, Gonfloni S, Ruberti F, Domenici L, Cattaneo A (1998) Eur J Neurosci 10:3127-3140.

Morisset V, Ahluwalia J, Nagy I, Urban L (2001) Eur J Pharmacol 429:93-100.

Nakatsuka T, Furue H, Yoshimura M, Gu J G (2002) J Neurosci 22:1228-1237.

Nilsson G, Forsberg-Nilsson K, Xiang Z, Hallbook F, Nilsson K, Metcalfe D D (1997) Eur J Immunol 27:2295-2301.

Porro C A, Cavazzuti M (1993) Spatial and temporal aspects of spinal cord and brainstem activation in the formalin pain model. Prog Neurobiol 41: 565-607.

Pesavento E, Margotti E, Righi M, Cattaneo A, Domenici L (2000) Neuron 25:165-175.

Ruberti F, Capsoni S, Cornparini A, Di Daniel E, Franzot J, Gonfloni S, Rossi G, Berardi N, Cattaneo A (2000) J Neurosci 20:2589-2601.

Saper C B, Gelman D C, White C L, 3rd (1985) Neurology 35:1089-1095.

Saragovi H U, Gehring K (2000) Trends Pharmacol Sci 21:93-98.

Sevcik M A, Ghilardi J R, Peters C M, Lindsay T H, Halvorson K G, Jonas B M, Kubota K, Kuskowski M A, Boustany L, Shelton D L, Mantyh P W (2005) Pain 115:128-141.

Shu X, Mendell L M (1999) Neurosci Lett 274:159-162.

Sivilotti L, Nistri A (1991) Prog Neurobiol 36:35-92.

Woolf C J, Ma Q P, Allchorne A, Poole S (1996) J Neurosci 16:2716-2723.

Zhu Z, Friess H, diMola F F, Zimmermann A, Graber H U, Korc M, Buehler M W (1999) J Clin Oncol 17:2419-2428.

1-24. (canceled)

25. An anti-NGF antibody capable of inhibiting binding between HGF ND TrkA for use in the treatment of chronic pain.

26. Method of treatment or prevention of chronic pain in a subject comprising administering to the subject an effective amount of an anti-NGF antibody capable of inhibiting binding between HGF ND TrkA to treat chronic pain in said subject.

27. (canceled)

28. Method of treatment or prevention of chronic pain as in claim 26 wherein the antibody is able to recognize and bind to a NGF molecule domain containing the aa. 41-49 region of human or rat NGF: EVNINNSVF (SEQ ID No. 9).

29. Method of treatment or prevention of chronic pain as in claim 26 wherein the antibody is able is able to recognize and bind to a NGF molecule domain containing the aa 23-35 region: GDKTTATDIKGKE (SEQ ID No. 10).

30. Method of treatment or prevention of chronic pain as in claim 26 wherein the antibody is capable of blocking the biological activity of TrkA.

31. Method of treatment or prevention of chronic pain as in claim 26 wherein the variable region of the antibody light chain comprises at least one of the complementarity determining regions (CDRs) having the sequence selected from aa. 24 to aa. 34 of SEQ ID No. 1; from aa. 50 to aa. 56 of SEQ ID No. 1; from aa. 89 to aa. 97 of SEQ ID No. 1.

32. Method of treatment or prevention of chronic pain as in claim 26 wherein the variable region of the antibody light chain comprises at least two of the complementarity determining regions (CDRs) having the sequence selected from aa. 24 to aa. 34 of SEQ ID No. 1; from aa. 50 to aa. 56 of SEQ ID No. 1; from aa. 89 to aa. 97 of SEQ ID No. 1.

33. Method of treatment or prevention of chronic pain as in claim 26 wherein the variable region of the antibody light chain comprises all of three complementarity determining regions (CDRs) having the sequence selected from aa. 24 to aa. 34 of SEQ ID No. 1; from aa. 50 to aa. 56 of SEQ ID No. 1; from aa. 89 to aa. 97 of SEQ ID No. 1.

34. Method of treatment or prevention of chronic pain as in claim 26 wherein the variable region of the antibody light chain comprises essentially the sequence of SEQ ID No. 1.

35. Method of treatment or prevention of chronic pain as in claim 26 wherein the variable region of the antibody heavy chain comprises at least one of the complementarity determining regions (CDRs) having the sequence selected from aa. 26 to aa. 35 of SEQ ID No. 2; from aa. 50 to aa. 65 of SEQ ID No. 2; from aa. 98 to aa. III of SEQ ID No. 2.

36. Method of treatment or prevention of chronic pain as in claim 26 wherein the variable region of the antibody heavy chain comprises at least two of the complementarity determining regions (CDRs) having the sequence selected from aa. 26 to aa. 35 of SEQ ID No. 2; from aa. 50 to aa. 65 of SEQ ID No. 2; from aa. 98 to aa. III of SEQ ID No. 2.

37. Method of treatment or prevention of chronic pain as in claim 26 wherein the variable region of the antibody heavy chain comprises all of three of the complementarity determining regions (CDRs) having the sequence selected aa. 26 to aa. 35 of SEQ ID No. 2; from aa. 50 to aa. 65 of SEQ ID No. 2; from aa. 98 to aa. III of SEQ ID No. 2.

38. Method of treatment or prevention of chronic pain as in claim 26 wherein the variable region of the antibody heavy chain comprises essentially the sequence of SEQ ID No. 2.

39. Method of treatment or prevention of chronic pain as in claim 26 wherein the antibody is in single chain form and comprises a light chain variable region and a heavy chain variable region joined by a linker.

40. Method of treatment or prevention of chronic pain as in claim 26 wherein the antibody comprises two light chains and two heavy chains.

41. Method of treatment or prevention of chronic pain as in claim 26 wherein the anti-NGF antibody is a human or humanized antibody.

42. Method of treatment or prevention of chronic pain as in claim 41 wherein the variable region of the humanized antibody light chain comprises essentially the sequence of SEQ ID No. 3.

43. Method of treatment or prevention of chronic pain as in claim 41 wherein the variable region of the humanized antibody heavy chain comprises essentially the sequence of SEQ ID No. 4.

44. Method of treatment or prevention of chronic pain as in claim 41 wherein the humanized antibody light chain has essentially the sequence of SEQ ID No. 8.

45. Method of treatment or prevention of chronic pain as in claim 41 wherein the humanized antibody heavy chain has essentially a sequence selected from SEQ ID No. 5, SEQ ID No. 6, SEQ ID No. 7.

46. Method of treatment or prevention of chronic pain as in claim 26 wherein the pain is of the chronic inflammatory type.

47. Method of treatment or prevention of chronic pain as in claim 46 wherein the chronic pain is caused by pancreatitis, kidney stones, headaches, dysmenorrhea, musculoskeletal pain, sprains, visceral pain, ovarian cysts, prostatitis, cystitis, interstitial cystitis, post-operative pain, migraine, trigeminal neuralgia, pain from burns and/or wounds, pain associated with trauma, neuropathic pain, pain associated with musculoskeletal diseases, rheumatoid arthritis, osteoarthritis, ankylosing spondilitis, periarticular pathologies, oncological pain, pain from bone metastases, pain from HIV.

48. Method of treatment or prevention of chronic pain as in claim 26 wherein the pain is neuropathic pain.

49. Method of treatment or prevention of chronic pain as in claim 26 wherein the pain is oncological pain.

50. Method of treatment or prevention of chronic pain as in claim 26 wherein the antibody has a long duration of action.

51. Use of an anti-NGF antibody capable of inhibiting the binding between NGF and TrkA for the preparation of a medicament for treating and/or preventing chronic pain.

52. A kit comprising a composition containing an antibody capable of inhibiting binding between NGF and TrkA together with instructions directing administration of said composition to a subject in need of treatment of chronic pain thereby to treat chronic pain in said subject.
